(19) United States
(12) Patent Application Publication
(10) Pub. No.: US 2008/0075676 A1
(43) Pub. Date: Mar. 27, 2008

(54) Dental Whitening Systems

(75) Inventors: Jeff MacDonald, Pomona, CA (US); Angela jinmin Lee, Glendale, CA (US); Nancy N. Quan, North Hills, CA (US)

Correspondence Address: DISCUS DENTAL IMPRESSIONS, INC., 8550 HIGUERA STREET, CULVER CITY, CA, 90232, US

(73) Assignee: Discus Dental, LLC, Culver City, CA (US)

(21) Appl. No.: 11/927,207

(22) Filed: Oct. 29, 2007

Related U.S. Application Data
(63) Continuation of application No. 11/271,283, filed on Nov. 9, 2005.
(60) Provisional application No. 60/626,407, filed on Nov. 9, 2004.
(60) Provisional application No. 60/631,121, filed on Nov. 26, 2004.
(60) Provisional application No. 60/643,309, filed on Jan. 13, 2005.
(60) Provisional application No. 60/653,421, filed on Feb. 15, 2005.

(51) Int. Cl. A61K 8/22

(52) U.S. Cl. 424/53

(57) ABSTRACT

A system and method for tooth whitening are disclosed wherein at least one peroxide-containing gel and at least one transition metal compound- containing gel, particularly at least one lower atomic number transition metal compound, more particularly at least a ferrous compound including gluconate, sulfate, nitrate, acetate or mixtures thereof, are applied to a patient's mouth. Gelling agent is also included. The activation of the peroxide whitens the patient's teeth. The system may be used with or without the application of light. The system further provides an additional gel including a sensitivity reduction compound including potassium nitrate, sodium nitrate or mixtures thereof for possible sensitivity treatment if needed.

### CROSS-REFERENCE TO RELATED APPLICATIONS

This application claims the benefit of U.S. Provisional patent applications Ser. Nos. 60/626,407, filed Nov. 9, 2004, entitled “Tooth Whitening Compositions”; 60/631,121, filed Nov. 26, 2004, entitled “Whitening System”; 60/643,309, filed Jan. 22, 2005 entitled “Dental Whitening”; and 60/653,421, filed Feb. 15, 2005, entitled “Whitening System Capable of Effective Whitening Action”; the contents of which are hereby incorporated by reference.

### FIELD OF INVENTION

This invention relates to tooth whitening compositions. More particularly, this invention relates to a two-component tooth whitening system.

### BACKGROUND OF INVENTION

A tooth is composed of an inner dentin layer an inner dentin layer and outer hard enamel that is coated with a protective layer called the acquired pellicle. The enamel layer of a tooth is composed of hydroxyapatite mineral crystals that create a somewhat porous surface. The pellicle or the enamel may become stained or discolored. It is believed that this porous nature of the enamel layer is what allows staining agents and discolor substances to permeate the enamel and discolor the tooth.

Many substances that a person's teeth confront or come in contact with on a daily basis can “stain” or reduce the “whiteness” of one's teeth. In particular, food products, tobacco products and fluids such as tea and coffee that one consumes tend to stain one's teeth. These staining and discoloring substances can then permeate the enamel and causing noticeable discoloration of one's teeth.

One solution to this problem is through tooth bleaching. Some dentrifices, like toothpastes, gels, and powders, contain active oxygen or hydrogen peroxide liberating bleaching agents including peroxides, percarbonates and perborates of the alkali and alkaline earth metals or complex compounds containing hydrogen peroxide.

Commonly used dental bleaching agent include hydrogen peroxide, carbamide peroxide (CO(NH₂)₂ H₂O₂), or urea hydrogen peroxide, hydrogen peroxide carbamide, and perhydrol-urea. Carbamides and hydrogen peroxides are used in over-the-counter compositions as well as bleaching gels are dispensed by dentists and commonly dispensed ones include those containing hydrogen peroxide (available as “DayWhite” from Discus Dental, Inc.) and those containing a mixture of hydrogen peroxide and carbamide peroxide (available as “NiteWhite”, also from Discus Dental, Inc.).

Some prior art discloses the use of concentrated carboxypolymethylene compositions for producing a matrix material having a sufficiently high viscosity, low solubility in saliva, and is sufficiently tacky to retain and hold a dental tray positioned over the patient's teeth for a period greater than about 2 hours without any significant mechanical pressure from the dental tray so as to provide for the dental bleaching agent to be in contact with the tooth surfaces thereby providing bleaching of the tooth surfaces.

The amount of whitening obtained during tooth bleaching is dependent upon (1) the length of time each day the tray is worn; (2) the number of days the tray is worn; (3) the susceptibility of the teeth to the bleaching agent and (4)concentration of active peroxide. For maximum whitening, an accelerated treatment time of approximately 18-20 hours per day is recommended.

One concern with some bleaching compositions is the prolonged period that is needed for effective bleaching. Another is the highly concentrated bleaching agents present in the composition. Both of these can contribute to tooth sensitivity following treatment. Accordingly, there is a need for a dental bleaching composition that has improved whitening action with normal or reduced amounts of bleaching agent and can produce effective bleaching at shorter times.

### SUMMARY OF THE INVENTION

The present invention discloses a two-component dental whitening system, a first component includes at least one peroxide compound, and a second component includes an orally compatible Activator including at least one transition metal compound. The transition metal compound catalyzes the whitening action of the peroxide compound to produce improved whitening at typical to lower peroxide concentrations.

The present invention also discloses a two-component dental whitening system, a first gel component includes at least one peroxide compound and a second component includes an orally compatible Activator Gel including at least one lower oxidative state transition metal compound. The metal compound catalyzes the whitening action of the peroxide compound to produce faster effective whitening at lower peroxide concentrations.

The present invention further discloses a two-component dental photobleaching system, a first gel component includes at least one peroxide compound and a second component includes an orally compatible Activator Gel including at least one lower oxidative state transition metal compound. The metal compound catalyzes the whitening action of the peroxide to produce faster effective whitening at typical or lower peroxide concentrations upon irradiating with light.

In one aspect of the invention, the peroxide compound is present, for example, from about 5% by weight to about 35% by weight of the first gel component.

In another aspect of the invention, the ratio of the Activator Gel component is, for example, from about a 1:2 to a 5:1.

In another aspect of the invention, the two components of the system may be provided in a two barrel syringe. In one embodiment, the syringe may be provided with a dispensing tip. In another embodiment, the dispensing tip is adapted for foaming. In a further embodiment, the tip may include a mixer.

In a further aspect of the invention, the transition metal compound is present at a concentration of from about 0.01% by weight to about 4% by weight of the Activator Gel component.

Any of the composition disclosed above may be in an unfoamed, foam or foamable state, all capable of delivering faster whitening action.

The present invention even further discloses a 2-component foamable composition having a first component including at least one peroxide compound; and a second component including an orally compatible Activator Gel including at least one transition metal compound and at least one foaming agent; wherein the two components combine to form a foam having a half life of at least about 10 minutes.

The transition metal compound catalyzes the whitening action of the peroxide to produce improved whitening at typical to lower peroxide concentrations.

In one embodiment, the 2-component foamable composition includes a first component including at least one peroxide compound in an aqueous solution; and a second component including at least one transition metal compound and at least one foaming agent in solid form.

In another embodiment, the 2-component foamable composition includes a first component including at least one peroxide compound in an aqueous solution and at least one peroxide stabilizer in the form of an ion scavenger; and a second component including at least one foaming agent and at least one transition metal compound peroxide activator; wherein said activator promotes the rapid decomposition of the peroxide compound and additional foaming action not related to the foaming agent.

Additionally, the present invention includes a light-activatable, 2-component foamable composition having a first component including at least one peroxide compound; and a second component including at least one foaming agent and one lower oxidative state transition metal compound; wherein the two components combine to form a foam having a half life of at least about 10 minutes.

The present invention still further includes a one-component, multi-phase, foamable composition including at least one peroxide compound in an aqueous phase and at least one transition metal compound and at least one foaming agent in an oil phase, wherein the two phases combine to form a foam having a half life of at least about 10 minutes.

In one aspect, a remineralizing agent may be present in any of the above compositions, to aid in remineralizing and de-sensitizing tooth or teeth.

The present invention additionally provides for a method for using the two-component dental whitening system, including the steps of:

(a) providing a first gel component including at least one peroxide;

(b) providing an Activator Gel including at least one transition metal compound capable of catalyzing the whitening action of the peroxide gel;

(c) applying the peroxide gel and the Activator Gel onto the teeth; and

(e) illuminating the teeth with light;

wherein said whitening system may be in an unfoamed, foamed or foamable state.

Furthermore, the present invention provides for a system of teeth whitening with sensitivity relief, including:

a two-component whitening gel including a first component gel includes at least one peroxide compound and a second component includes an orally compatible Activator Gel including a transition metal compound that catalyzes the whitening action of the peroxide to produce effective whitening at typical or lower peroxide concentrations; and
a one-component viscous gel including between about 3% to about 5% by weight of alkali metal salt, such as potassium nitrate and a source of fluoride ion at a concentration of up to about 1100 ppm, said gel having a viscosity comparable to that of the two-component whitening gel.

The present invention together with the above and other advantages may best be understood from the following detailed description of the exemplary embodiments of the invention.

### DETAILED DESCRIPTION OF THE INVENTION

The detailed description set forth below is intended as a description of the presently exemplified tooth whitening composition provided in accordance with aspects of the present invention and is not intended to represent the only forms in which the present invention may be prepared or utilized. The description sets forth the features and the steps for preparing and using the tooth whitening compositions of the present invention. It is to be understood, however, that the same or equivalent functions and ingredients incorporated in the tooth whitening compositions may be accomplished by different embodiments that are also intended to be encompassed within the spirit and scope of the invention.

Unless defined otherwise, all technical and scientific terms used herein have the same meaning as commonly understood to one of ordinary skill in the art to which this invention belongs. Although any methods, devices and materials similar or equivalent to those described herein may be used in the practice or testing of the invention, the exemplified methods, devices and materials are now described.

All publications mentioned herein are incorporated herein by reference for the purpose of describing and disclosing, for example, the compositions and methodologies that are described in the publications which might be used in connection with the presently described invention. The publications listed or discussed above, below and throughout the text are provided solely for their disclosure prior to the filing date of the present application. Nothing herein is to be construed as an admission that the inventors are not entitled to antedate such disclosure by virtue of prior invention.

The coronal portion of the tooth consists of enamel, dentin and the pulp. In the mouth of humans, the enamel is coated with an acquired pellicle. The tooth structures that are generally responsible for presenting a stained appearance are enamel, dentin, and the acquired pellicle. Tooth enamel is predominantly formed from inorganic material, mostly in the form of hydroxyapatite crystals, and may further contain approximately 5% organic material primarily in the form of collagen. In contrast, dentin is composed of about 20% protein including collagen, the balance consisting of inorganic material, predominantly hydroxyapatite crystals, similar to that found in enamel. The acquired pellicle is a proteinaceous layer or matrix that forms continuously over the surface of the tooth. Although the acquired pellicle may be removed through intensive mechanical cleaning, it quickly regenerates soon thereafter.

Discoloration of teeth may result from intrinsic and/or extrinsic staining. Intrinsic staining occurs when staining compounds penetrate the enamel and even the dentin, or alternatively, such staining arises from sources within the tooth. Typically such staining may only be removed through chemical methods of tooth cleaning.

As noted above, the amount of whitening obtained during tooth whitening is generally dependent upon (1) the length of time the teeth is in contact with the whitening agent; (2) the number of days the treatment is carried out; (3) the susceptibility of the teeth to the whitening agent and (4)concentration of active peroxide. For maximum whitening, a long treatment time with a highly concentrated whitening composition is generally recommended. Such treatments usually take place in a dentist's office as highly concentrated whitening compositions are not recommended for unsupervised home use. However, extended treatment times are also not amenable for office visits. Even treatments with concentrated whitening compositions followed by irradiation with a light source generally take more than an hour per visit to accomplish. For take home treatments, a relatively low concentration of whitening composition followed by extended hours of treatment time still do not produce the same good results as office treatments.

The present inventors have invented a whitening composition including a two-component dental whitening composition, including a first gel including at least one peroxide compound (the “peroxide gel”), and at least one transition metal compound containing gel (the “Activator Gel”) that catalyzes the whitening action. The resulting composition has superior whitening properties at typical or lower peroxide concentration.

The present invention is also directed to a two-component dental whitening composition that generates faster effective whitening at shorter whitening times when compared with other photoactivatable whitening compositions.

The exemplary peroxide gel includes metal ion free peroxide compounds. Examples of suitable metal ion free peroxide compounds include hydrogen peroxide and organic peroxides including urea peroxide (carbamide peroxide), glyceryl peroxide, benzoyl peroxide and the like. In general, hydrogen peroxide, carbamide peroxide and mixtures thereof are used, with hydrogen peroxide being the more generally used. The total peroxide present in the gel, for example, ranges form about 5% by weight to about 35% by weight of the peroxide gel, more for example, from about 10% by weight to about 32% by weight of the peroxide gel, even more for example, from about 12% by weight to about 25% by weight of the peroxide-containing gel.

When hydrogen peroxide is used, it is usually provided as a 50% aqueous solution. When used alone, the amount ranges, for example, from about 10% to about 60% (5% to 30% in the absence of water), more for example, the amount ranges from about 20% to about 40% (10% to 20% in the absence of water).

On the other hand, when carbamide peroxide is used, it is, for example, used in combination with hydrogen peroxide in an amount from about 9% to about 20%, more for example, from about 5% to about 16%. At the same time, if hydrogen peroxide is also present, it is for example, provided as a 50% aqueous solution and is generally present in an amount of from about 5% to about 50% (2.5% to 25% in the absence of water).

As mentioned above, the composition of the present invention may be an unfoamed, foamed or foamable composition. In general, a foamed composition may include the same or higher peroxide concentration as that present in an unfoamed composition. On the other hand, a foamable composition may include a higher peroxide concentration in the unfoamed state so that the concentration of peroxide after foaming may be the same or higher than the level present in a typical gel. For foamable compositions, the amounts of peroxide noted above represent those in the foamed state.

Sequestering agents such as salts of ethylene diamine tetraacetic acid, diethylene triamine pentaacetic acid, phosphonates such as Dequest (trademark) available from Monsanto Chemical Company and azacycloheptane 2′,2′ diphosphonate are generally used as chelating agents to stabilize the peroxide containing compositions by chelating metal ions such as Fe (III), Mn(II), and Cu(II) that might be present in the peroxide-containing compositions. Surprisingly, when a small amount of a transition metal compound is present in the Activator Gel component of the whitening composition, the compound acts as a catalyzing agent for the peroxide gel component of the whitening composition so that when the peroxide compound or compounds is present in typical or even lower concentrations generally used in a dentist's office, the time needed for producing the same whitening effect is reduced by, for example, at least about 15%, more for example, at least about 20%, and eve more for example, at least about 25%.

The exemplary transition metals are those of lower atomic numbers including lower atomic number transition metals such as those ranging from atomic number 21 to 30. Also, useful ones include those with lower oxidative states, for example, iron(II), manganese(II), cobalt(II), copper(II) and mixtures thereof, and more for example, Iron(II).

Without wishing to be bound by a particular theory, it is surmised that the above mentioned transition metal compounds participate in either a Fenton reaction or a photoFenton reaction, in which a lower oxidative state transition metal, such as Fe(II), may react with peroxides such as hydrogen peroxide, either thermally or upon exposure to light, to facilitate the dissociation of peroxide into active whitening species such as hydroxyl radicals, perhydroxy anions or superoxide radicals, as exemplified below:
H₂O₂+Fe²⁺→Fe³⁻+HO⁻+HO*

Surprising, only a very small amount of transition metal compound is needed, for example, from about 0.01% by weight to about 4% by weight, more for example, from about 0.03% by weight to about 2% by weight, and even more for example, from about 0.04% to about 1% by weight, as the lower oxidative state transition metal is regenerated in the whitening action, as exemplifies below:
HO*+Fe^{3+→HO}⁺+Fe²⁻

It is further surmised that when the transition metal compounds are at their most effective when they remain in solution and do not precipitate out of the gel. Thus, anions may be chosen so that the transition metal compounds have good solubility in the Activator Gel component after mixing with the peroxide gel. Those anions may include gluconates, sulfates, nitrates, acetates and mixtures thereof. It is surmised also that the chelating effect of an anion does not play any role in the catalyzing action, as chelating action tends to stabilize the peroxides, rather than to catalyze their decomposition. Thus, though gluconates are capable of chelating with transition metals, are present merely to aid in solubilizing the transition metals, and any chelating effect may be negligible.

The amounts of transition metal compounds present in the Activator Gel may also affect their solubility. Soluble compounds tend to be better catalysts. Thus, it is surmised that useful transition metal compounds do not precipitate out of solution, as noted above. Fortunately, small amounts of such compounds are effective. The effect of their solubility and low concentration of transition metal compounds act to improve the whitening action of the peroxide gel as noted above.

Other ingredients may be added to improve both the peroxide and gel stability. These include gelling agents, gel stabilizers, surfactants, any compound having a strongly ionic component, pH adjusting agents, humectants, and other adjuvants for improving gel consistency may be added to both the peroxide gel and the Activator Gel component.

Examples of gelling agents include non-ionic surfactants such as those having both a hydrophilic component and a hydrophobic component, including copolymers of ethylene oxide and propylene oxide. The copolymers may be block copolymers of propylene oxide (hydrophobic component), and ethylene oxide (hydrophilic component). The propylene oxide block may generally be sandwiched between two ethylene oxide blocks. Literally hundreds of versions of such non-ionic surfactants having both hydrophobic and hydrophilic blocks are possible by incremental alteration of both hydrophobe and hydrophile. In addition, heteric or alternating ethylene oxide/propylene oxide structures may be introduced internally or at the end of the molecule. The generally used ones include Pluronic F-127, P-84 or mixtures thereof, available from BASF Corporation (North Mount Olive, N.J., USA).

Other gelling agents suitable for use in the preparation of both peroxide and Activator Gels may include, for example, other non-ionic surfactants, such as the classes that limit the number of available hydrophobes and effect changes in surfactant function only by altering the hydrophile, are suitable.

Still other gelling agents which may be used in the preparation of whitening gels may include, for example, cellulosic gums, fumed silica, for example, CAB-O-SIL fumed silica provided by Cabot Corporation, and emulsifying waxes such as Polawax (emulsifying wax NF) or Crodafos CES (cetearyl alcohol (and) dicetyl phosphate (and) ceteth-10 phosphate), provided by Croda, Inc., and mixtures thereof, in amounts to provide a stable gel. Some examples of cellulosic gum may include ‘Klucel’GF, a hydroxymethyl propylcellulose from Hercules.

The amount of surfactants present in the gel may determine whether a gel can or cannot form as well as the gel's stability. With too little gelling agent, no gel may form. With too much gelling agent, no gel formation is possible. Stable gels are formed when the gelling agents are present within the appropriate range. Exemplary percent of hydrophobic components in the gelling agents ranges, for example, from about 10 to about 80, more for example, from about 30 to about 60, even more for example, from about 31 to about 38, and still even more for example, from about 33 to about 36.

For example, the non-ionic surfactant may be present in an amount that provides a stable dental peroxide gel and may, for example, range from about 15% to about 45% by weight of the peroxide gel, more for example, from about 20% by weight to about 40% by weight. In the Activator Gel component, the non-ionic surfactant may be present, for example, from about 15% to about 30%, more for example, from about 20% to about 25% by weight.

The total molecular weight may also be varied. For example, molecular weights may range from about 300 to about 20,000, more for example, from about 5,000 to about 12,000.

Other ingredients that may also be added to aid the stability of a gel may include any compound having a strongly ionic component, as mentioned above. Exemplary ones may include alkali compounds, for example, potassium compounds (such as potassium nitrate), sodium compounds (such as sodium nitrate), vitamin E oil, anise, eugenol, natural mint flavors or mixtures thereof. Less favored ones, though still effective, may include heavy metal halides, especially chlorine-containing compounds.

For example, an insoluble stabilizer such as vitamin E oil, anise, eugenol, natural mint flavors and mixtures thereof may be added to help the stability of the gel formation in both components of the whitening gel.

The total weight % of the stabilizer varies from, for example, about 0.25 to about 5, more for example, from about 0.5 to about 4, even more for example, from about 1 to about 3 in the peroxide gel; and for example, from about 1 to about 20, more for example, from about 2 to about 15, and even more for example, from about 4 to about 12 in the Activator Gel.

Alkali metals such as potassium and sodium compounds, including potassium nitrate, when added, may be present at the higher end of the range to stabilize the peroxide gel and increase gel shelf life. Some, such as potassium nitrate, may also increase the sensitivity relief of the gel, as further discussed below.

For example, the peroxide gel may also include a source of calcium, strontium or combinations thereof. The source of calcium, strontium or combinations thereof in the peroxide component may include, for example, a calcium compound, a strontium compound, or mixtures thereof, more for example, a calcium compound such as calcium nitrate, in an amount of, for example, from about 0.25% by weight to about 1.5% by weight, more for example, about 0.3% to about 1% by weight.

When calcium compound is present in the peroxide-containing gel, it is useful that the Activator Gel also contains a source of, for example, phosphate. For example, the source of phosphate in the Activator Gel may include monosodium phosphate (NAH₂PO₄), disodium phosphate, tetrapotassium pyrophosphate or mixtures thereof. The source of phosphate may be, for example, present in an amount of from about 0.2% to about 5% by weight, more for example, between about 0.2% to about 4% by weight.

When the two gel components are mixed, combines with phosphate to form the various amorphous calcium and/or strontium phosphates.

Amorphous calcium compounds such as amorphous calcium phosphate (ACP), amorphous calcium phosphate fluoride (ACPF), amorphous calcium carbonate phosphate (ACCP), amorphous calcium carbonate phosphate (ACCP), and amorphous calcium carbonate phosphate fluoride (ACCPF) are useful for aiding in remineralizing teeth. These amorphous compounds are disclosed in U.S. Pat. Nos. 5,037,639, 5,268,167, 5,437,857, 5,562,895, 6,000,341, and 6,056,930, the disclosure of each is hereby incorporated by reference in its entirety.

In addition to amorphous calcium compounds, amorphous strontium compounds such as amorphous strontium phosphate (ASP), amorphous strontium phosphate fluoride (ASPF), amorphous strontium calcium phosphate (ASCP), amorphous strontium calcium carbonate phosphate (ASCCP), amorphous strontium carbonate phosphate fluoride (ASCPF) and amorphous strontium calcium carbonate phosphate fluoride (ASCCPF) may be used in re-mineralization, as noted above. These compounds are disclosed in U.S. Pat. No. 5,534,244, the content of which is hereby incorporated by reference in its entirety.

When applied to the teeth, amorphous calcium phosphate or others mentioned above may precipitate onto the surface of the teeth where it may be incorporated into hydroxyapatite, assisting in remineralization of the tooth enamel, as is also discussed in U.S. Pat. Nos. 5,037,639, 5,268,167, 5,460,803, 5,534,244, 6,000,341, and 6,056,930, incorporated herein by reference.

Some of the compounds mentioned above may also be used in fluoridating teeth. All of the above amorphous compounds or solutions which form the amorphous compounds, when applied either onto or into dental tissue, may in addition, aid to prevent and/or repair dental weaknesses such as dental caries, exposed roots and dentin sensitivity.

Surprisingly, the amorphous calcium and/or strontium compounds present in the composition may also act as sensitivity relief agents. In fact, the present inventors have found that the de-sensitizing effect provided by amorphous calcium phosphate is at least as effective as, if not more effective than, the typical de-sensitizing agents normally used, including some of those discussed below. Therefore, the presence of amorphous calcium and/or strontium compounds may potentially replace traditional de-sensitizing agents.

In practice, as much phosphate as possible or practicable may be included. However, amounts of monosodium phosphate in excess of about 4% by weight may tend to affect gel stability.

Surprisingly, the phosphate component present in the first component according to the ranges mentioned above may also act to stabilize the gel. The levels may be present from, for example, about 0.5% to about 5% by weight, more for example, from about 1% to about 4% by weight. At higher levels, the stabilizing effect gradually disappears. For monosodium phosphate, amounts in excess of about 4% by weight may tend to affect gel stability.

Additionally, the phosphate compound may further act to adjust the pH of the first component. The pH of the system is, for example, from about 5 to about 8, more for example, from about 5.5 to about 6.5.

In one embodiment of the present invention, the Activator Gel may have, for example, a pH of from about 2 to about 7; more for example, from about 3 to 6, and even more for example, from about 3 to about 5.5. To keep the transition metal compounds from precipitating out of solution, a lower pH of the Activator Gel may be used. However, some compounds may be added to keep the metal compound from precipitating out even at higher pH. These may include glyconic acid, gallic acid or mixtures thereof. Gallic acid is generally used as a high pH additive.

As noted, the lower the pH of the Activator Gel, the less likely the transition metal compounds will precipitate out. However, lower pH may contribute to demineralization of teeth. In the present invention, a pH of the composition as low as about 1 to about 3 may be used by virtue of the addition of amorphous calcium phosphate or others mentioned above, the shorter exposure time and the lower concentration of the peroxide. Any one or combinations of these factors may aid to reduce the effect of demineralization.

Additional components added to improve gel consistency and stability may be added to one or both components.

According to the manufacturer of the PLURONIC™ products, glycols such as propylene glycol and ethylene glycol are not recommended as they tend to degrade the PLURONIC™ gels formed. Therefore, it is not desirable to include any propylene glycol, and humectants in the peroxide gel normally do not contain any glycols.

Surprisingly, in the formulation of the Activator Gel, glycols such as propylene glycol, polyethylene glycol or mixtures thereof may actually aid in the formation and stability of the gels. Amounts, for example, from about 5% by weight to about 20% by weight, more for example, about 10% to about 15% by weight may be used. Other non-toxic glycols may also used or be present in addition.

When the two-components are mixed, any glycols present in the Activator Gel surprisingly do not affect the stability of the combined gel and the whitening gel may be applied as if no glycols were present.

Exemplary gel modifying polyols for use in the peroxide-containing gel also include aliphatic polyols in an amount, for example, from about 1% by weight to about 10% by weight, more for example, from about 2% to about 5% by weight. An exemplary polyol may be glycerine (Merck Index #4493, 12th Ed.), present, for example, in an amount up to about 2% by weight. For the Activator Gel, an aliphatic polyol such as glycerine may be present in an amount, for example, from about 2% by weight to about 15% by weight, more for example, from about 4% to about 12% by weight.

In addition to the addition of heat, light and/or chemicals, the amount of whitening obtained during a whitening process is generally dependent upon (1) the length of time the teeth is in contact with the whitening agent; (2) the number of days the treatment is carried out; (3) the susceptibility of the teeth to the whitening agent and (4) concentration of active peroxide, as noted above. For maximum whitening, a long treatment time with a highly concentrated whitening composition is generally recommended, as noted before.

Whitening activity of a peroxide compound is generally dictated by the availability of active peroxides, and not by the actual concentration of peroxide present in the composition. When peroxide is present in solution, active peroxides are readily available. However, a solution, by its nature, is not easily contained, and/or not amenable for sustain action when applied to a patient's teeth, again because it is difficult to confine it any desired location for any length of time. Thus, a less concentrated peroxide solution requiring longer contact time to be effective is not a practical solution environment. A more concentrated solution of peroxide, though more efficient in whitening, is likewise not suitable in a solution environment because it will not solve the confinement and prolonged contact problem. In addition, any concentrated peroxide solution that may come into contact with soft tissue inside a patient's mouth may potentially cause tissue damage. Therefore, to maintain effective bleaching with good containment so as to minimize potential tissue damage, various gelling agents, thickeners, adhesion promoters and/or similar additives may be used, as discussed above in the unfoamed system. These result in the formation of paste, gels, and similar forms, which are effective whitening systems. It is surmised that some of the additives used, though effective in containment and bleaching, may somewhat decrease the bleaching activity by inhibiting the availability of active peroxides, especially if the composition contain thickeners or adhesion promoters derived from polymers of acrylic acid (carbomer), pyrrolidone analog thickeners, or others. Such additives may lead to diminished whitening capacity of peroxides through ionic and covalent interactions within the gel, and act against the desired effect of tooth whitening.

Foaming may be another way to improve substantivity without some of the above mentioned additives. Foamed compositions may also provide prolonged contact without high amounts of additives that may tend to somewhat inhibit the activity of active peroxides, thus further improving the whitening activity. Foams may be formulated with the same or higher amounts of active ingredients and smaller amounts of inactive ingredients. In a given volume, smaller amounts of inactive ingredients such as gelling agents, thickeners, adhesion promoters or similar may be present in the foamed state compared to unfoamed state. Without wishing to be bound to a theory, it is surmised that air performs the function of an inactive ingredient such as a gelling agent, a thickener, an adhesion promoter or mixtures thereof, to give the needed substantivity; but air molecules in general do not inhibit the availability of active peroxides to the same extent as other inactive ingredients needed for substantivity in an unfoamed gel. Thus, a foamed composition has the substantivity of a composition having higher amounts of ingredients such as gelling agents, thickeners, adhesion promoters or similar inactive ingredients, but with more availability of active peroxides for bleaching action even if the same concentration of peroxide is present. In other words, foaming may in effect substitute air bubbles for gelling agents to create substantivity.

Thus, a foamed or foamable composition of the present invention may increase the rate of whitening activity, if desired, without the problems encountered by solutions. Not only is a foamed composition manageable without confinement problems during use, it may also be capable of delivering a high concentration of active peroxide by not inhibiting the availability of active peroxides, and at the same time, may also seek to fill crevices, sometimes seeping into gaps where gaps are not apparent, thus offering effective whitening action not only to the front surface of a tooth, but surfaces in-between teeth as well. Therefore, foaming may potentially provide all the benefits that are not currently available to other whitening systems.

Foams in general also have lower surface tension than their unfoamed counterparts and may therefore be spread as thin as one molecule thick sheets, while at the same time increasing its surface area, thus covering more area with the same volume of whitening composition than their unfoamed counterparts. The ability to lower surface tension may also make the stains easier to remove.

The foamed bubbles, through the forces of capillary action and lowered surface tension, are also themselves good wetting agents, so that a bleaching solution may penetrate through smaller openings, as noted above. The rapid penetration into the tightest spots may also be aided by the distribution of the sizes and shapes of bubbles. Thus, foams may even cause deeper penetration of active peroxide molecules.

It is further surmised that active peroxides may also be captured inside the bubbles of foams. As the bubbles collapse, the active peroxide agent is released to perform whitening actions.

Foam formation may be effected by any agitation or whipping action, use of foaming agents and combinations thereof. In some embodiments, foaming agents may be present to render the composition foamable so that foams are generated just prior to use. Some foaming agents may also aid in increasing the adhesion of the gel to a solid surface by allowing it to spread over a greater surface area. In others embodiments, foams are formed just prior to use by agitation with or without any foaming agent.

Thus, some compositions may include at least one foaming agent. Different categories of foaming agents are suitable, and they may produce foams in different ways. Suitable foaming agents may include certain surfactants such as anionic, nonionic, amphoteric, zwitterionic, cationic, or mixtures thereof.

Some of these aids in foam formation and some do not. Some surfactants are useful purely for their foaming properties alone, some act only as emulsifiers or wetting agents without foaming, and some even act to reduce foaming. The functions of the surfactants are not necessarily determined by the category they belong to.

Anionic surfactants include, but not limited to water-soluble compounds of alkyl sulfates having from about 8 to about 20 carbon atoms in the alkyl radical (such as sodium alkyl sulfate), water-soluble compounds of sulfonated monoglycerides of fatty acids having from about 8 to about 20 carbon atoms and mixtures thereof. Examples of anionic surfactants include Sodium lauryl sulfate, sodium coconut monoglyceride sulfonates, phospholipids, sarcosinates, such as sodium lauryl sarcosinate, taurates, sodium lauryl sulfoacetate, sodium lauroyl isethionate, sodium laureth carboxylate, and sodium dodecyl benzenesulfonate. Many of these anionic surfactants are disclosed in U.S. Pat. No. 3,959,458, the content of which is incorporated herein in its entirety by reference.

Nonionic surfactants may include, but not limited, to compounds including a hydrophilic and hydrophobic components (which maybe produced by the condensation of alkylene oxide groups (hydrophilic in nature) with an organic hydrophobic compound which may be aliphatic or alkyl-aromatic in nature). Examples of suitable nonionic surfactants include low viscosity poloxamers (sold under the trade name Pluronic), low viscosity hydroxyethyl cellulose, polysorbates, polyoxyethylene sorbitan esters (sold under the trade name Tweens), fatty alcohol ethoxylates, polyethylene oxide condensates of alkyl phenols, products derived from the condensation of ethylene oxide with the reaction product of propylene oxide and ethylene diamine, ethylene oxide condensates of aliphatic alcohols, long chain tertiary amine oxides, long chain tertiary phosphine oxides, long chain dialkyl sulfoxides, and mixtures thereof.

Amphoteric surfactants may include, but is not limited to derivatives of aliphatic secondary and tertiary amines in which the aliphatic component may be a straight chain or branched and one of the aliphatic substituents contains from about 8 to about 18 carbon atoms and one contains an anionic water-solubilizing group, such as carboxylate, sulfonate, sulfate, phosphate, phosphonate, betaines, specifically cocamidopropyl betaine, and mixtures thereof.

Many of these nonionic and amphoteric surfactants are disclosed in U.S. Pat. No. 4,051,234, the content of which is incorporated herein by reference in its entirety.

In the present invention, the exemplified surfactants, when used in foamable compositions, are those that not only have foaming capabilities, but also those with the ability to act as wetting agents.

Actually, any asymmetrical molecule dissolved in water will make at least a weak surfactant. Such weak surfactants may normally not be an effective foaming agent, but its effectiveness may be improved if a foaming dispenser is used. Asymmetrical molecules as used herein include those that include a hydrophilic and a hydrophobic segment, such as some of the nonionic surfactants mentioned above. One end of the molecule is thus polar in nature and dissolves in water, while the other end is nonpolar in nature and avoids water. When in water, the surfactant molecules oriented themselves with their polar ends towards the water molecules, leaving the nonpolar ends free to attract nonpolar molecules. It is surmised that in a foamed or foamable composition of the present invention, the non-polar ends help to lift tooth surface stains, allowing them to be washed away with the water.

The amount of foaming agents may range, for example, from about 0.1% to about 5% by weight of the foamable composition, more for example, from about 0.5% to about 3% by weight, even more for example, less than about 1% by weight.

Still other foaming agents may include reaction products of any base with an acid. These may include, for example, an alkali metal carbonate or bicarbonate, such as sodium bicarbonate, potassium bicarbonate, sodium carbonate or potassium carbonate, or an alkaline metal carbonate or bicarbonate such as magnesium or calcium bicarbonate or carbonate. The amount used may range, for example, from about 1% to about 10% by weight, more for example, from about 3% to about 7%, still more for example, from about 3.5 to about 5.5% by weight of the composition. The amount used may also depend on the volume of foam required. Thus, by varying the amount of foaming agents, the amount of foam produced may be varied accordingly.

Generally, the ratio of acid and base ranges, for example, from about 1:0.5 to 1:25, more for example, from about 1:1 to 1:4, by weight. Suitable acids include strong acids such as water soluble carboxylic acids, phosphoric acids, nitric acids, and/or sulfuric acids.

Additionally, a foaming agent may also be a gaseous material. The gaseous material may be any inert gas or a gas generated by mixing a basic peroxide solution with an acid solution.

Since the whitening activity results when active peroxide comes into contact with the tooth, the foams generated just prior to use may for example, not need to be too long lasting, if the active ingredients are surmized to be captured within the bubbles, but have a substantive body so that the amount of liquid formed, i.e., the collapsing of the foam bubbles to release active peroxide, may be controlled and balanced. Therefore, the exemplary compositions are not only foamable, but are also capable of producing longer lasting, collapsible foams.

The foams generated by a foamable composition generally have half lives of, for example, at least about 10 minutes, more for example, from about 10 minutes to about 120 minutes. A foam having a half life of 10 minutes means that 50% of the bubbles would collapse to release the encapsulated whitening agent, or that the volume of the foam is reduced by 50% in about 10 minutes after formation, and 75% of all the bubbles are gone, or the volume of the foam is reduced by 75% in about 20 minutes.

The collapse time or half lives of the foam depends on a number of factors. For example, the greater the amount of bubbles formed, the longer the collapse time. Also, the lower the viscosity of the thickeners, and/or other inactive ingredients, the shorter the collapse time. The collapse time may also depend on the nature of the other additives to the composition, which have surface active properties e.g., surfactants or preservatives as well as the environment. For example, a drier environment may contribute to faster collapse of bubbles.

For foamed compositions, the collapsible bubbles typically have very extended half lives when confined in the package, for example, at least about a month, more for example, at least about three months. During use, the collapse of the foamed bubbles is aided by the environment, such as the loss of water or solvent to the environment, and the saliva in the patient's mouth. A packaging may be designed so that a foamed composition may have a desired shelf life after the package or container has been opened and the remaining has been exposed to air. A single-use packaging may also be designed.

For example, a lower viscosity gelling agent or thickener may be used. They are not as likely to inhibit the availability of active peroxides to the same extent as a higher viscosity gelling agent. The viscosity is for example, generally less than about 10,000 cps, more for example, less than about 8,000 cps, and even more for example, less than about 5,000 cps.

Suitable foaming agents include foamable surfactants including at least some sodium lauryl Sulfate as the primary foaming agent.

As discussed above, substantivity, i.e. the ability of a product to linger, is a desirable property in any whitening composition. On the other hand when the desired property of a product is the ability to be rinsed off easily, a foaming surfactant would not be used. However, there is a general desire that a whitening composition may both have substantivity and the ease to be rinsed off. When this is desired, foams generated in a “foaming pump”, a dispensing tip adapted for foaming, or a dispensing tip including a mixer adapted for foaming may be used. Such foaming devices again may produce foams with desirable properties, while using a minimum of amount of surfactants, for example, less than about 0.5%. At the same time, some combinations of additives may be chosen to produce the desired effect as well, such as by the addition of other foaming agents.

The gels may be prepared using any known method. Typically, in preparing the peroxide gel, the peroxide compound, stabilizing polyols such as glycerin, and water sufficient to produce the desired concentration of peroxide are completely mixed, then any compounds, such as calcium nitrate and/or potassium nitrate, are added and mixed at high speed until completely dissolved. A gelling agent such as PLURONIC® F-127 is added at high speed until the gelling agent is completely dispersed and the resulting composition is almost whipped in texture.

Eugenol and/or natural mint flavorings, preferably undiluted, are next added, resulting in a thickening of the whitening gel. The gel may be mixed at ambient temperatures for about thirty to forty minutes, or until the desired consistency is achieved, then vacuum degassed.

Alternatively, following addition of eugenol and/or mint flavorings, the peroxide gel may be stored at temperatures sufficiently low to liquify the gelling agent, approximately 10° F when PLURONIC® F-127 is used, allowing air trapped in the gel to rise to the surface, where it may be scrapped off as foam or removed by vacuum degassing.

For peroxide gels, water is present in an amount of, for example, between 10% to about 20%, more for example, from about 13% to about 16% by weight. In the Activator Gels, water is present, for example, from about 30% to about 70% by weight, more for example, from about 40% to about 60% by weight.

Both components of the whitening system may be substantially free of abrasives.

Non-limiting examples of peroxide gels provided in accordance with practice of the present invention are as follows:

Peroxide Gel1

| Material | % by weight | Grams (300 g total) |
|---|---|---|
| Water | 15.5 | 46.5 |
| Glycerin | 2.0 | 6 |
| 50% H₂O₂ (in water) | 40.0 | 120 |
| Ca(NO3)2 | 0.5 | 1.5 |
| KNO₃ | 1.0 | 3.0 |
| PLURONIC ® F-127 | 40.5 | 121.5 |
| Eugenol | 0.25 | 0.75 |
| Natural Mint | 0.25 | 0.75 |

Peroxide Gel 2

| Material | % by weight | Grams (300 g total) |
|---|---|---|
| Water | 13.65 | 40.95 |
| Glycerin | 2.0 | 6.0 |
| 50% H₂O₂ (in water) | 50.0 | 150 |
| Ca(NO3)2 | 0.5 | 1.5 |
| KNO₃ | 2.0 | 6.0 |
| PLURONIC ® F-127 | 30.0 | 90.0 |
| Eugenol | 0.75 | 2.25 |
| Natural Mint | 1.1 | 3.3 |

Peroxide Gel 3

| Material | Trade name | % | Grams |
|---|---|---|---|
| Water | | 0.00 | 0 |
| Glycerin | | 4.65 | 9.3 |
| Hydrogen Peroxide 50% CG | | 64.00 | 128 |
| Poly dimethyl siloxane | Antifoam 1520-US | QS | QS |
| Poloxomer 407 | Pluronic F-127 | 28.00 | 56 |
| Eugenol | | 0.75 | 1.5 |
| Natural Peppermint Oil | | 1.10 | 2.2 |
| KNO3 | | 1.50 | 3 |
| Total | | 100.00 | |

Peroxide Gel 4

| Material | Trade name | % | Grams |
|---|---|---|---|
| Water | | 2.65 | 5.3 |
| Glycerin | | 2.00 | 4 |
| KNO3 | | 1.50 | 3 |
| Hydrogen Peroxide 50% CG | | 64.00 | 128 |
| Poly dimethyl siloxane | Antifoam 1520-US | 0.00 | 0 |
| Poloxomer 407 | Pluronic F-127 | 28.00 | 56 |
| Eugenol | | 0.75 | 1.5 |
| Natural Peppermint Oil | | 1.10 | 2.2 |
| | | 100.00 | |

Peroxide gel 5

| Material | % | Grams |
|---|---|---|
| Water | 27.75 | 55.5 |
| Polysorbate 80 | 0.5 | 1 |
| SLS | 0.25 | 0.5 |
| Glycerin | 2 | 4 |
| 50% H2O2 | 28 | 56 |
| Pluronic F-127 | 41 | 82 |
| Eugenol | 0.25 | 0.5 |
| Natural Mint | 0.25 | 0.5 |
| | 100 | 200 |

Peroxide Gel 6

| Material | % | Grams |
|---|---|---|
| Phosphoric Acid | 0.25 | 0.75 |
| Polysorbate 80 | 0.5 | 1.5 |
| Water | 11.5 | 34.5 |
| Sodium Laurel Sulfate | 0.25 | 0.75 |
| 50% H2O2 | 48 | 144 |
| Pluronic F-127 | 39 | 117 |
| Eugenol | 0.25 | 0.75 |
| Natural Mint | 0.25 | 0.75 |
| | 100 | |

Peroxide Gel 7

| Material | % | Grams |
|---|---|---|
| Water | 26.5 | 79.5 |
| Glycerin | 2 | 6 |
| 50% H2O2 | 40 | 120 |
| Ca(NO3)2 | 0.5 | 1.5 |
| Potassium Nitrate | 1 | 3 |
| Pluronic F-127 | 28 | 84 |
| Eugenol | 0.75 | 2.25 |
| Natural Mint | 1.25 | 3.75 |
| | 100 | 300 |

Peroxide Gel 8

| Material | % | Grams |
|---|---|---|
| Water | 15 | 45 |
| Glycerin | 2 | 6 |
| 50% H2O2 | 40 | 120 |
| Ca(NO3)2 | 0.5 | 1.5 |
| Potassium Nitrate | 1 | 3 |
| Pluronic F-127 | 40.5 | 121.5 |
| Natural Mint | 1 | 3 |
| | 100 | 300 |

Peroxide Gel 9

| Material | % | Grams |
|---|---|---|
| Water | 14.25 | 42.75 |
| Glycerin | 2 | 6 |
| 50% H2O2 | 50 | 150 |
| Ca(NO3)2 | 0.5 | 1.5 |
| KNO3 | 2 | 6 |
| Pluronic F-127 | 30 | 90 |
| Natural Mint | 1.25 | 3.75 |
| | 100 | 300 |

Peroxide Gel 10

| Material | % | Grams |
|---|---|---|
| Water | 15.5 | 46.5 |
| Glycerin | 2 | 6 |
| 50% H2O2 | 40 | 120 |
| Ca(NO3)2 | 0.5 | 1.5 |
| Potassium Nitrate | 1 | 3 |
| Pluronic F-127 | 40.5 | 121.5 |
| Eugenol | 0.25 | 0.75 |
| Natural Mint | 0.25 | 0.75 |
| Total | 100 | 300 |

Peroxide Gel 11

| Material | % | Grams |
|---|---|---|
| Water | 22 | 66 |
| Glycerin | 2 | 6 |
| 50% H2O2 | 40 | 120 |
| Ca(NO3)2 | 0.5 | 1.5 |
| Pluronic F-127 | 35 | 105 |
| Eugenol | 0.25 | 0.75 |
| Natural Mint | 0.25 | 0.75 |
| Total | 100 | 300 |

Peroxide Gel 12

| Material | % | Grams |
|---|---|---|
| Water | 18.65 | 55.95 |
| Glycerin | 2 | 6 |
| 50% H2O2 | 45 | 135 |
| Ca(NO3)2 | 0.5 | 1.5 |
| KNO3 | 2 | 6 |
| Pluronic F-127 | 30 | 90 |
| Eugenol | 0.75 | 2.25 |
| Natural Mint | 1.1 | 3.3 |
| Total | 100 | 300 |

Peroxide Gel 13

| Material | % | Grams |
|---|---|---|
| Water | 14.9 | 44.7 |
| Glycerin | 2 | 6 |
| 50% H2O2 | 48.75 | 146.25 |
| Ca(NO3)2 | 0.5 | 1.5 |
| KNO3 | 2 | 6 |
| 10% KOH | QS | |
| Pluronic F-127 | 30 | 90 |
| Eugenol | 0.75 | 2.25 |
| Natural Mint | 1.1 | 3.3 |
| Total | 100 | 300 |

Peroxide Gel 14

| Material | % | Grams |
|---|---|---|
| Water | 14.9 | 44.7 |
| Glycerin | 2 | 6 |
| 50% H2O2 | 48.75 | 146.25 |
| Ca(NO3)2 | 0.5 | 1.5 |
| KNO3 | 2 | 6 |
| 10% KOH | QS | |
| Pluronic F-127 | 30 | 90 |
| Eugenol | 0.75 | 2.25 |
| Natural Mint | 1.1 | 3.3 |
| Total | 100 | 300 |

Peroxide Gel 15

| Material | % | Grams |
|---|---|---|
| Water | 13.65 | 40.95 |
| Glycerin | 2 | 6 |
| 50% H2O2 | 50 | 150 |
| Ca(NO3)2 | 0.5 | 1.5 |
| KNO3 | 2 | 6 |
| Pluronic F-127 | 30 | 90 |
| Eugenol | 0.75 | 2.25 |
| Natural Mint | 1.1 | 3.3 |
| Total | 100 | 300 |

Peroxide Gel 16

| Material | % | Grams |
|---|---|---|
| Water | 18.65 | 55.95 |
| Glycerin | 2 | 6 |
| 50% H2O2 | 45 | 135 |
| Ca(NO3)2 | 0.5 | 1.5 |
| KNO3 | 2 | 6 |
| Pluronic F-127 | 30 | 90 |
| Eugenol | 0.75 | 2.25 |
| Natural Mint | 1.1 | 3.3 |
| Total | 100 | 300 |

Peroxide Gel 17

| Material | Trade name | % | Grams |
|---|---|---|---|
| Water | | 9.65 | 19.3 |
| Glycerin | | 2.00 | 4 |
| KNO3 | | 1.50 | 3 |
| Ca(NO3)2 | | 1.00 | 2 |
| Hydrogen | CG | 56.00 | 112 |
| Peroxide 50% | | | |
| Poly dimethyl | Antifoam | 0.00 | 0 |
| siloxane | 1520-US | | |
| Poloxomer 407 | Pluronic F-127 | 28.00 | 56 |
| Eugenol | | 0.75 | 1.5 |
| Natural | | 1.10 | 2.2 |
| Peppermint Oil | | | |
| Total | | 100.00 | |

Peroxide Gel 18

| Material | % | Grams |
|---|---|---|
| Water | 16 | 48 |
| Glycerin | 2 | 6 |
| 50% H2O2 | 40 | 120 |
| Ca(NO3)2 | 0.5 | 1.5 |
| Pluronic F-127 | 41 | 123 |
| Eugenol | 0.25 | 0.75 |
| Natural Mint | 0.25 | 0.75 |
| Total | 100 | 300 |

Peroxide Gel 19

| Material | % | Grams |
|---|---|---|
| Water | 16 | 32 |
| Glycerin | 2 | 4 |
| 50% H2O2 | 40 | 80 |
| TKPP | 0.5 | 1 |
| Pluronic F-127 | 41 | 82 |
| Eugenol | 0.25 | 0.5 |
| Natural Mint | 0.25 | 0.5 |
| Total | 100 | 200 |

Peroxide Gel 20

| Material | % | Grams |
|---|---|---|
| Water | 27.75 | 55.5 |
| Polysorbate 80 | 0.5 | 1 |
| SLS | 0.25 | 0.5 |
| Glycerin | 2 | 4 |
| 50% H2O2 | 28 | 56 |
| Pluronic F-127 | 41 | 82 |
| Eugenol | 0.25 | 0.5 |
| Natural Mint | 0.25 | 0.5 |
| Total | 100 | 200 |

Peroxide Gel 21

| Material | % | Grams |
|---|---|---|
| Water | 28.5 | 57 |
| Glycerin | 2 | 4 |
| 50% H2O2 | 28 | 56 |
| Pluronic F-127 | 41 | 82 |
| Eugenol | 0.25 | 0.5 |
| Natural Mint | 0.25 | 0.5 |
| Total | 100 | 200 |

Peroxide Gel 22

| Material | % | Grams |
|---|---|---|
| Water | 31.5 | 63 |
| Glycerin | 2 | 4 |
| 50% H2O2 | 25 | 50 |
| Pluronic F-127 | 41 | 82 |
| Eugenol | 0.25 | 0.5 |
| Natural Mint | 0.25 | 0.5 |
| Total | 100 | 200 |

Peroxide Gel 23

| Material | % | Grams |
|---|---|---|
| Water | 24.5 | 49 |
| Glycerin | 2 | 4 |
| 50% H2O2 | 32 | 64 |
| Pluronic F-127 | 41 | 82 |
| Eugenol | 0.25 | 0.5 |
| Natural Mint | 0.25 | 0.5 |
| Total | 100 | 200 |

Peroxide Gel 24

| Material | % | Grams |
|---|---|---|
| Water | 16.5 | 33 |
| Glycerin | 2 | 4 |
| 50% H2O2 | 40 | 80 |
| Pluronic F-127 | 41 | 82 |
| Eugenol | 0.25 | 0.5 |
| Natural Mint | 0.25 | 0.5 |
| Total | 100 | 200 |

Peroxide Gel 25

| Material | % | Grams |
|---|---|---|
| Phosphoric Acid | 0 | 0 |
| Polysorbate 80 | 0 | 0 |
| Water | 18.5 | 37 |
| Sodium Laurel | 0 | 0 |
| Sulfate | | |
| 50% H2O2 | 40 | 80 |
| Pluronic F-127 | 41 | 82 |
| Eugenol | 0.25 | 0.5 |
| Natural Mint | 0.25 | 0.5 |
| | 100 | 200 |

Peroxide Gel 26

| Material | % | Grams |
|---|---|---|
| Phosphoric Acid | 0 | 0 |
| Polysorbate 80 | 0.5 | 1 |
| Water | 25.75 | 51.5 |
| Sodium Laurel | 0.25 | 0.5 |
| Sulfate | | |
| 50% H2O2 | 32 | 64 |
| Pluronic F-127 | 41 | 82 |
| Eugenol | 0.25 | 0.5 |
| Natural Mint | 0.25 | 0.5 |
| Total | 100 | 200 |

Peroxide gel 27

| Material | % | Grams |
|---|---|---|
| Phosphoric Acid | 0.25 | 0.5 |
| Polysorbate 80 | 0.5 | 1 |
| Water | 17.5 | 35 |
| Sodium Laurel | 0.25 | 0.5 |
| Sulfate | | |
| 50% H2O2 | 40 | 80 |
| Pluronic F-127 | 41 | 82 |
| Eugenol | 0.25 | 0.5 |
| Natural Mint | 0.25 | 0.5 |
| Total | 100 | 200 |

Peroxide Gel 28

| Material | % | Grams |
|---|---|---|
| Water | 27.25 | 81.75 |
| Glycerin | 2 | 6 |
| 50% H2O2 | 40 | 120 |
| Ca(NO3)2 | 0.5 | 1.5 |
| Potassium | 1 | 3 |
| Nitrate | | |
| Pluronic F-127 | 28 | 84 |
| Eugenol | 0 | 0 |
| Natural Mint | 1.25 | 3.75 |
| Total | 100 | 300 |

An exemplary method for preparing the Activator Gel is as follows: In preparing the Activator Gel, the aqueous solution of ferrous compound is preferably combined with potassium nitrate and monosodium phosphate, plus an amount of water sufficient to produce the desired concentration of components in the Activator Gel, and mixed until all components are completely dissolved. The mixture is then titrated with a pH adjusting agent, such as disodium phosphate, to the desired pH.

Glycerine and/or other polyols are added to the mixture along with propylene glycol, and mixed until dissolved. The gelling agent is then whipped into the mixture at high speed, producing a stiff gel. Finally, any natural mint flavors, such as natural peppermint oil, are whipped into the gel, further stiffening of the Activator Gel. The gel is mixed at ambient temperatures for about thirty to forty minutes, or until the desired consistency is achieved, then vacuum degassed at about 75-100 Torr, for example, at less than 100 Torr.

Non-limiting examples of Activator Gels provided in accordance with practice of the present invention are as follows:

Activator Gel 1

| Material | % by weight |
|---|---|
| Water | 55.65 |
| Potassium nitrate | 2.5 |
| Ferrous gluconate (premix with | 0.042 |
| water) | |
| Water (premix with ferrous | 1.208 |
| gluconate) | |
| Monosodium phosphate | 3.75 |
| Disodium phosphate | Qs to pH 5.3-5.4 |
| Propylene glycol | 11.1 |
| Glycerin | 4.25 |
| PLURONIC ® F-127 | 20.5 |
| Natural Peppermint Oil | 1.0 |

Activator gel 2

| | | Grams |
|---|---|---|
| Material | % by wt | (200 g total) |
| Water | 54.65 | 109.3 |
| KNO₃ | 5.25 | 10.5 |
| NaH₂PO₄ | 2 | 4 |
| Na₂HPO₄ | QS | QS to pH 5.1-5.3 |
| 3.33% Fe Gluc. in | 1 | 2 |
| water | | |
| Antifoam | QS | QS |
| Propylene Glycol | 11.1 | 22.2 |
| Glycerin | 4.5 | 9 |
| PLURONIC ® F-127 | 20.5 | 41 |
| Natural Mint | 1 | 2 |

Activator Gel 3

| | | Grams |
|---|---|---|
| Material | % by wt | (200 g total) |
| Water | 51.55 | 103.1 |
| KNO₃ | 3 | 6 |
| NaH₂PO₄ | 2 | 4 |
| Na₂HPO₄ | QS | QS to pH 5.1-5.3 |
| 3.33% Fe Gluc. in water | 1.25 | 2.5 |
| Antifoam | QS | QS |
| Propylene Glycol | 11.1 | 22.2 |
| Glycerin | 9.6 | 19.2 |
| PLURONIC ® F-127 | 20.5 | 41 |
| Natural Mint | 1 | 2 |

Activator Gel 4

| | | Grams |
|---|---|---|
| Material | % by wt | (200 g total) |
| Water | 49.55 | 99.1 |
| KNO₃ | 5 | 10 |
| NaH₂PO₄ | 2 | 4 |
| Na₂HPO₄ | QS | QS to pH 5.1-5.3 |
| 3.33% Fe Gluc. in water | 1.25 | 2.5 |
| Antifoam | QS | QS |
| Propylene Glycol | 11.1 | 22.2 |
| Glycerin | 9.6 | 19.2 |
| PLURONIC ® F-127 | 20.5 | 41 |
| Natural Mint | 1 | 2 |

Activator Gel 5

| | | Grams |
|---|---|---|
| Material | % by wt | (200 g total) |
| Water | 47.55 | 95.1 |
| KNO₃ | 7 | 4 |
| NaH₂PO₄ | 2 | 4 |
| Na₂HPO₄ | QS | QS to pH 5.1-5.3 |
| 3.33% Fe Gluc. in water | 1.25 | 2.5 |
| Antifoam | QS | QS |
| Propylene Glycol | 11.1 | 22.2 |
| Glycerin | 9.6 | 19.2 |
| PLURONIC ® F-127 | 20.5 | 41 |
| Natural Mint | 1 | 2 |

Activator Gel 6

| | | Grams |
|---|---|---|
| Material | % by wt | (200 g total) |
| Water | 47.55 | 95.1 |
| KNO₃ | 7 | 14 |
| NaH₂PO₄ | 2 | 4 |
| Na₂HPO₄ | QS | QS to pH 5.1-5.3 |
| 3.33% Fe Gluc. in water | 1.25 | 2.5 |
| Antifoam | QS | QS |
| Propylene Glycol | 11.1 | 22.2 |
| Glycerin | 9.6 | 19.2 |
| PLURONIC ® F-127 | 20.5 | 41 |
| Natural Mint | 1 | 2 |

Activator Gel 7

| | | Grams |
|---|---|---|
| Material | % by wt | (200 g total) |
| Water | 49.55 | 99.1 |
| KNO₃ | 3 | 6 |
| KCl | 2 | 4 |
| NaH₂PO₄ | 2 | 4 |
| Na₂HPO₄ | QS | QS to pH 5.1-5.3 |
| 3.33% Fe Gluc. in water | 1.25 | 2.5 |
| Antifoam | QS | QS |
| Propylene Glycol | 11.1 | 22.2 |
| Glycerin | 9.6 | 19.2 |
| PLURONIC ® F-127 | 20.5 | 41 |
| Natural Mint | 1 | 2 |

Activator Gel 8

| | | Grams |
|---|---|---|
| Material | % by wt | (200 g total) |
| Water | 47.55 | 95.1 |
| KNO₃ | 5 | 10 |
| KCl | 2 | 4 |
| NaH₂PO₄ | 2 | 4 |
| Na₂HPO₄ | QS | QS to pH 5.1-5.3 |
| 3.33% Fe Gluc. in water | 1.25 | 2.5 |
| Antifoam | QS | QS |
| Propylene Glycol | 11.1 | 22.2 |
| Glycerin | 9.6 | 19.2 |
| PLURONIC ® F-127 | 20.5 | 41 |
| Natural Mint | 1 | 2 |

Activator Gel 9

| | | Grams |
|---|---|---|
| Material | % by wt | (200 g total) |
| Water | 47.55 | 95.1 |
| KNO₃ | 3 | 6 |
| KCl | 4 | 8 |
| NaH₂PO₄ | 2 | 4 |
| Na₂HPO₄ | QS | QS to pH 5.1-5.3 |
| 3.33% Fe Gluc. in | 1.25 | 2.5 |
| water | | |
| Antifoam | QS | QS |
| Propylene Glycol | 11.1 | 22.2 |
| Glycerin | 9.6 | 19.2 |
| PLURONIC ® F-127 | 20.5 | 41 |
| Natural Mint | 1 | 2 |

Activator Gel 10

| | | Grams |
|---|---|---|
| Material | % by wt | (200 g total) |
| Water | 45.55 | 91.1 |
| KNO₃ | 5 | 10 |
| KCl | 4 | 8 |
| NaH₂PO₄ | 2 | 4 |
| Na₂HPO₄ | QS | QS to pH 5.1-5.3 |
| 3.33% Fe Gluc. in | 1.25 | 2.5 |
| water | | |
| Antifoam | QS | QS |
| Propylene Glycol | 11.1 | 22.2 |
| Glycerin | 9.6 | 19.2 |
| PLURONIC ® F-127 | 20.5 | 41 |
| Natural Mint | 1 | 2 |

Activator Gel 11

| | | Grams |
|---|---|---|
| Material | % by wt | (200 g total) |
| Water | 43.55 | 87.1 |
| KNO₃ | 7 | 14 |
| KCl | 4 | 8 |
| NaH₂PO₄ | 2 | 4 |
| Na₂HPO₄ | QS | QS to pH 5.1-5.3 |
| 3.33% Fe Gluc. in | 1.25 | 2.5 |
| water | | |
| Antifoam | QS | QS |
| Propylene Glycol | 11.1 | 22.2 |
| Glycerin | 9.6 | 19.2 |
| PLURONIC ® F-127 | 20.5 | 41 |
| Natural Mint | 1 | 2 |

Activator Gel 12

| | | Grams |
|---|---|---|
| Material | % by wt | (200 g total) |
| Water | 52.55 | 105.1 |
| KNO₃ | 3 | 6 |
| NaH₂PO₄ | 2 | 4 |
| Na₂HPO₄ | QS | QS to pH 5.1-5.3 |
| 3.33% Fe Gluc. in | 1.25 | 2.5 |
| water | | |
| Antifoam | QS | QS |
| Propylene Glycol | 11.1 | 22.2 |
| Glycerin | 9.6 | 19.2 |
| PLURONIC ® F-127 | 20.5 | 41 |
| Natural Mint | 1 | 2 |

Activator Gel 13

| | | Grams |
|---|---|---|
| Material | % by wt | (200 g total) |
| Water | 50.55 | 101.1 |
| KNO₃ | 3 | 6 |
| NaH₂PO₄ | 2 | 4 |
| Na₂HPO₄ | QS | QS to pH 5.1-5.3 |
| 3.33% Fe Gluc. in | 1.25 | 2.5 |
| water | | |
| Antifoam | QS | QS |
| Propylene Glycol | 11.1 | 22.2 |
| Glycerin | 9.6 | 19.2 |
| PLURONIC ® F-127 | 20.5 | 41 |
| Natural Mint | 2 | 4 |

Activator Gel 14

| | | Grams |
|---|---|---|
| Material | % by wt | (200 g total) |
| Water | 57.55 | 115.1 |
| KNO₃ | 3 | 6 |
| NaH₂PO₄ | 2 | 4 |
| Na₂HPO₄ | QS | QS to pH 5.1-5.3 |
| 3.33% Fe Gluc. in | 1.25 | 2.5 |
| water | | |
| Antifoam | QS | QS |
| Propylene Glycol | 8.1 | 16.2 |
| Glycerin | 6.6 | 13.2 |
| PLURONIC ® F-127 | 20.5 | 41 |
| Natural Mint | 1 | 2 |

Activator Gel 15

| | | Grams |
|---|---|---|
| Material | % by wt | (200 g total) |
| Water | 45.55 | 91.1 |
| KNO₃ | 3 | 6 |
| NaH₂PO₄ | 2 | 4 |
| Na₂HPO₄ | QS | QS to pH 5.1-5.3 |
| 3.33% Fe Gluc. in | 1.25 | 2.5 |
| water | | |
| Antifoam | QS | QS |
| Propylene Glycol | 14.1 | 28.2 |
| Glycerin | 12.6 | 25.2 |
| PLURONIC ® F-127 | 20.5 | 41 |
| Natural Mint | 1 | 2 |

Activator Gel 16

| | | Grams |
|---|---|---|
| Material | % by wt | (200 g total) |
| Water | 49.55 | 99.1 |
| KNO₃ | 3 | 6 |
| NaCl | 2 | 4 |
| NaH₂PO₄ | 2 | 4 |
| Na₂HPO₄ | QS | QS to pH 5.1-5.3 |
| 3.33% Fe Gluc. in | 1.25 | 2.5 |
| water | | |
| Antifoam | QS | QS |
| Propylene Glycol | 11.1 | 22.2 |
| Glycerin | 9.6 | 19.2 |
| PLURONIC ® F-127 | 20.5 | 41 |
| Natural Mint | 1 | 2 |

Activator Gel 17

| | | Grams |
|---|---|---|
| Material | % by wt | (200 g total) |
| Water | 47.55 | 95.1 |
| KNO₃ | 3 | 6 |
| NaCl | 4 | 8 |
| NaH₂PO₄ | 2 | 4 |
| Na₂HPO₄ | QS | QS to pH 5.1-5.3 |
| 3.33% Fe Gluc. in | 1.25 | 2.5 |
| water | | |
| Antifoam | QS | QS |
| Propylene Glycol | 11.1 | 22.2 |
| Glycerin | 9.6 | 19.2 |
| PLURONIC ® F-127 | 20.5 | 41 |
| Natural Mint | 1 | 2 |

Any or all of the above formulation, or any of the previously described compositions may be foamed. In some embodiments, a foaming agent may be added.

In packaging the tooth whitening composition of the present invention, any convenient means for effecting the separation of the peroxide gel from the Activator Gel before use may be utilized. For example, a single container may be compartmentalized so that the peroxide gel and the Activator Gel are housed in separate compartments and are dispensed simultaneously and admixed prior to application on the teeth. Alternatively, the peroxide gel and the Activator Gel may be housed in separate containers from which the respective phases are dispensed for admixture just prior to use. An exemplary packaging is disclosed in U.S. Pat. Nos. 5,819,988, 6,065,645, 6,394,314, 6,564,972 and 6,698,622, incorporate herein by reference. Also, the dispenser may be fitted with an agitator or pump for foaming.

In any of the packaging methods described, the syringe or the dual-compartment container may be provided with a dispensing tip, or a dispensing tip including a mixer. The mixers may be dynamic or static. Examples of static mixers may include those also disclosed in U.S. Pat. Nos. 5,819,988, 6,065,645, 6,394,314, 6,564,972 and 6,698,622, incorporated herein by reference. Examples of some dynamic mixers may include those disclosed in U.S. Pat. Nos. 6,443,612, and 6,457,609; and U.S. Patent Publication No. 2002/0190082; the contents of these are hereby incorporated by reference.

One exemplary embodiment of the present invention, the two components may be provided in separate chambers of a dual barrel syringe. Immediately before use, the two components are mixed together in, for example, a 1:2 to a 5:1 ratio (peroxide gel to Activator Gel) by actuating the syringe, more for example, the gels are mixed in the 4:1 ratio. The admixed whitening gel is applied to the surface of the teeth directly from the syringe. Other combinations of the peroxide gel and Activator Gel are contemplated by the present invention, depending on the desired final concentration of peroxide in the whitening gel. Typically, the peroxide content of the whitening gel is, for example, from about 5% to about 45%, more for example, about 10% to about 40%, as noted above.

Exemplary methods of using the whitening composition include those involving the use of light to activate the whitening compositions. One exemplary method is described in detail in U.S. Ser. Nos. 10/715,681, entitled “Tooth Whitening Process”, filed Nov. 17, 2003; 11/173839 “Illumination System For Dentistry Applications”, filed Jun. 30, 2005; 11/173371 entitled “Support Structure for Dental Applications” filed Jun. 30 , 2005, the entire contents of which are hereby incorporated by reference.

Preliminarily, if the peroxide gel and/or the whitening gel have been refrigerated, it is recommended that both are brought to room temperature prior to use to reconstitute a viscous gel. Upon combination of the peroxide gel and the Activator Gel, the resultant whitening gel may be sufficiently stiff so that it may remain on the teeth when applied. Typically, the gel(s) is, for example, removed from refrigeration at least six hours before use, and more for example, the night before, to allow it to reach room temperature. Alternatively, the refrigerated gels may be brought to room temperature more quickly by gently heating them as, for example, by submerging the container holding the gels in hot water (about 120° F./49° C.) for approximately ten minutes. If the gel(s) is/are heated in this manner, it is customary to wait about 5 minutes before combining the gels and applying them to the teeth, and care is taken to ensure that the temperature of the gels do not cause discomfort or injury to the patient when applied to the teeth.

Briefly, the first stage of the method in accordance with aspects of the present invention may involve isolation of the teeth and protection of the non-tooth surfaces that might otherwise be exposed to the whitening composition in the absence of protection. Optionally, a commercially available protective lip cream is applied to the lips to protect the lips. A cotton swab may be used to coat the lips with the cream. The cream is configured to keep the lips moist during the procedure and provide added protection from light exposure. Exemplary protective lip creams include paba free creams with high SPF rating, of about 30 or higher.

Next, a lip retractor, such as the Zoom!™ Retractor from Discus Dental, Inc., of Culver City, Calif., is installed to pull the lips away from the teeth. For additional isolation, a dentist may optionally apply medical grade petroleum jelly in the upper and lower vestibules, and cover the area with 2″×2″ four-ply gauze squares (Banta Healthcare Group), cotton rolls, or other suitable materials having suitable size to cover any remaining exposed tissues within the oral cavity.

A protective material may be applied to the gingiva to protect the gums from exposure to the whitening composition and the light radiation to be applied thereon. Preferably, a light-cured dental resin, such as Discus Dental's Liquidam™ Dental Dam, is applied and cured. The gingiva is generally dried prior to application of the protective material. The protective materials, which may generally be a light curable resin-based material, may be syringed directly onto the gingiva with sufficient amount for full gingival protection. The application extends distally for at least one tooth beyond the area to receive the whitening application. The application also may extend up or down to meet the gauze or retractor cover to protect the margins. Once the application of the dental dam is complete, the margins are rechecked to ensure that the dam is sealed against the enamel to prevent leakage and oxidation of tissue during the whitening procedure.

Immediately before use, the peroxide gel and the Activator Gel may be mixed together to form the whitening gel. The whitening gel is applied directly to the surface of the teeth as, for example, from the tip of a dual barrel syringe. The gel is applied to an approximate depth of about 1-2 mm. A brush may be used to ensure proper placement of the gel. Alternatively, the whitening composition may be dispensed into a dappen dish or into a mixing pad for placement onto the tooth surface with a brush. If contact occurs between the whitening gel and tissue during application of the whitening composition, Vitamin E Oil may be applied to the oxidized tissue. The oil material will help sooth the tissue and create a barrier so that the whitening procedure may continue.

Once the whitening gel is applied to the teeth, a lamp, such as a short arc metal halide lamp, is directed at the patient's smile zone to illuminate the whitening gel and thereby activate the whitening process. An exemplary suitable lamp is the SMR-150UV1, Model No. 04-1001, from Ushio America, Inc., of Cypress, Calif.

Sometimes filters may be placed in front of the lamp to screen out unwanted or harmful wavelengths. Suitable filters include a UV filter (available from Optical Industrial of Houston, Tex., Part No. 03-1013), an IR filter (available from Swift Glass of Elmira, N.Y., part No. 03-1017), and/or a diffuser filter (available from Edmund Industrial Optics of Barrington, N.J., part No. 03-1020).

The IR filter may be selected to filter all IR wavelengths to thereby reduce heat emitted to the patient. The UV filter may also be selected to remove some, but not all, of the ultraviolet wavelengths. Also, UV wavelengths below about 345 nm are filtered by the UV filter and only about 10-15% of wavelengths above about 400 nm are allowed to pass through the filter. The diffuser filter may be configured to diffuse the light rays and minimize the intensity of light irradiating onto the target area of the mouth. In one exemplary embodiment, the edge of the lamp may be positioned about 118 mm from the diffuser filter.

Once the lamp is secured in place and the smile zone illuminated, the whitening cycle may begin by activating the light. For the compositions of the instant invention, this will involve an approximately, for example, a 10- to 20-minute, more for example, a 15-minute, cycle. If the light assembly has not been used recently, a warm up cycle of a few minutes may be initiated. At the end of the cycle, the gel is suctioned from the patient's teeth and any remaining gel removed with a damp gauze.

For the whitening compositions of the present invention and light assembly described, a second and a third application of the whitening gel and light activating cycle may be used. However, more cycles may be used if necessary to achieve the desired degree of whitening.

After the final cycle has been completed, the lamp is removed and the remaining whitening gel is suctioned and wiped from the teeth. The isolation materials (gauze, cotton rolls, gingival protection) are then removed and, with the retractor in place, the oral cavity is thoroughly rinsed and suctioned. Dental floss may be used to remove any dental resin material that remains interproximally.

As noted above, compositions of the present invention requires about 15% less time to produce the same whitening effect for an identical or even higher peroxide content whitening composition without the transition metal compound catalyst. Some representative test results are shown below:

With the retractor still in place, various post-treatment procedures may optionally be carried out. For example, the facial area of the teeth may be dried and a fluoride solution (such as 1.1% Neutral Sodium Fluoride) may be applied, for example, by syringing it directly onto the teeth, approximately 2-3 mm thick, and allowing it to remain on the teeth for about 5 minutes before suctioning and rinsing.

As noted before, the whitening system of the present invention can perform faster whitening employing typical or lower levels of peroxide content compared to one without the transition metal compound or compounds. An exemplary side by side comparison, using the same whitening protocol was carried out between a gel with and without a transition metal compound and the results are shown below:

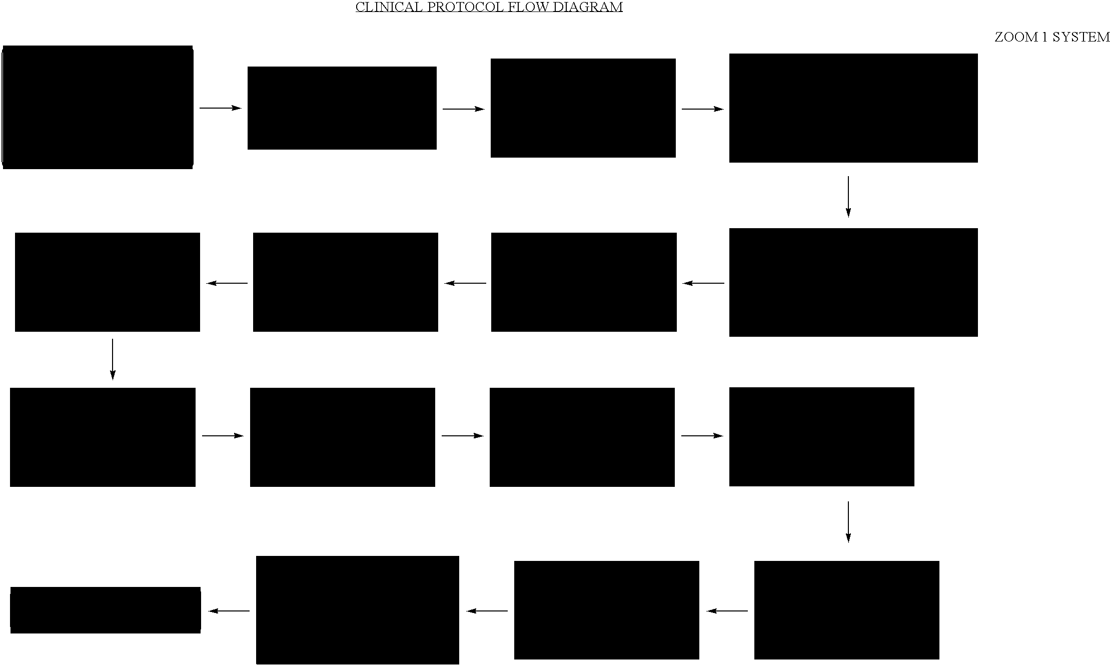

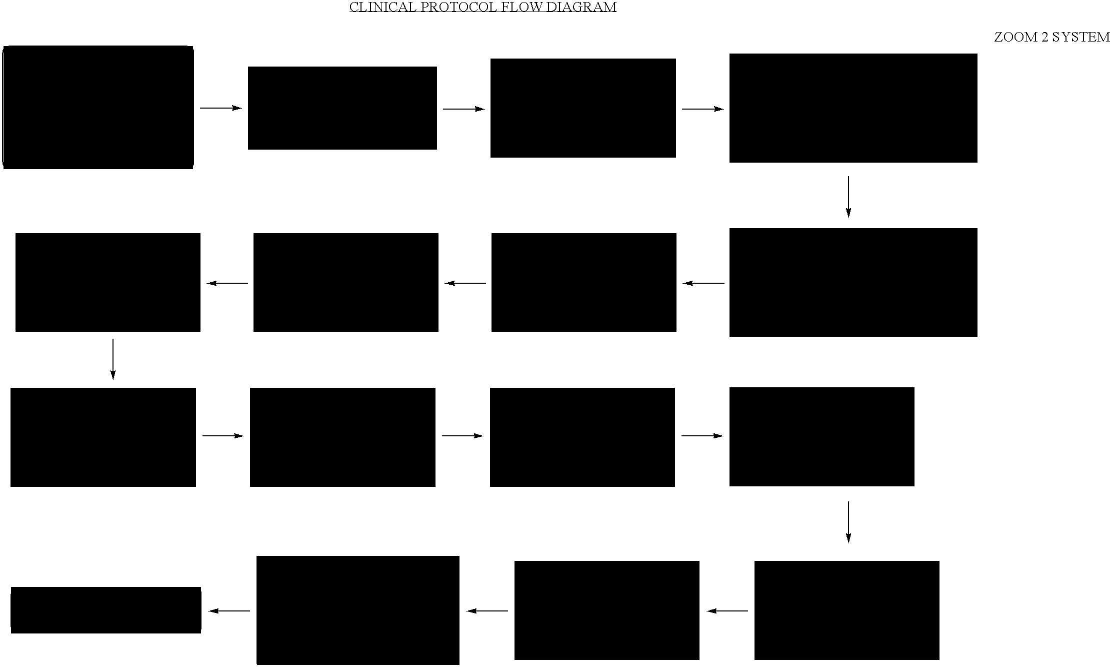

For the Zoom 2 composition (identical to the Zoom 1 composition, with the addition of ferrous gluconate), the time required to achieve equivalent whitening is reduced form 20 minutes to 15 minutes for each of the “Zoom Light TX” (light exposure) steps.

A foamed system may be applied according to the same method described above. For a foamed system, the amount of time needed for producing similar effects as a non-foamed system may be shortened.

As noted above, with the present invention, shorter time is required to effect teeth whitening, though typical or lower concentration of peroxide is present in the composition. In addition, the amorphous calcium or strontium phosphate may also aid in sensitivity relief. However, even at the reduced time of application and reduced peroxide concentrations and amorphous phosphate compounds, some patients may still experience teeth sensitivity after treatment. For those patients, a separate gel treatment may be provided. This de-sensitizing gel includes potassium nitrate desensitizer gels including a dissolved sodium fluoride at a concentration of, for example, between about 500 ppm to about 1500 ppm, more for example, from about 800 to about 1100 ppm and dissolved potassium nitrate, for example, at about 2.5% to about 6% by weight, more for example, from about 3% to about 5% by weight. The gelling matrix is formed primarily with non-ionic surfactants including ethylene oxide block co-polymers, as described above in the gel formation of the peroxide gel or Activator Gel, for example, PLURONIC™ F-127. The gelling agent is, for example, present from about 20% to about 40% by weight of the gel, more for example, from about 25% to about 35% of the gel.

Other ingredients may be present. These include gel stabilizers such, or any of the above mentioned ingredients useful in the whitening system. The stabilizers are present, for example, from about 0.1% to about 8%, more for example, from about 1% to about 5% by weight.

Aliphatic polyols such as glycerine may also be added in the amount of, for example, about 0% to about 10%, more for example, from about 1% to about 4% by weight.

Water is added in an amount of, for example, from about 50% to about 70%, more for example, from about 55% to about 65%.

The product may be applied generally after a whitening session, either at the dentist office or at home in a similar manner as that used for applying the whitening gel described above. It may be applied directly to a patient's teeth for a clinically relevant time period, usually 5-15 minutes and then suctioned off. An alternative means of application is by means of “night guard” form fitting tooth trays.

There is a significant improvement in the handling characteristics of the gel in that it is more easily applied by the dentist, and significantly more easily removed than many currently available products. Improved ease of use translates into faster procedures, reduced “office time” and a potential for reduced cost, increased profitability for the dental office. A second advantage could be the increased wetting ability of this composition and reduced gumminess that may indicate an increased availability of active ingredients and a possible improvement in overall efficacy.

The de-sensitizing gel may be made in the same manner as the above peroxide and/or Activator Gels. A representative formulation of this de-sensitizing gel is exemplified below:

| Material | Trade Name | % | Grams |
|---|---|---|---|
| Water | | 62.66 | 125.32 |
| Glycerin | | 2.00 | 4 |
| KNO3 | | 5.00 | 10 |
| Sodium Fluoride | | 0.243 | 0.486 |
| Sodium Saccharin | | 0.25 | 0.5 |
| Poloxomer 407 | Pluronic F-127 | 28.00 | 56 |
| Eugenol | | 0.75 | 1.5 |
| Natural Peppermint | | 1.10 | 2.2 |
| Oil | | | |
| Total | | 100.00 | |

While this invention is described in detail with reference to a certain preferred embodiments, it should be appreciated that the present invention is not limited to those precise embodiments. Rather, in view of the present disclosure which describes the current best mode for practicing the invention, many modifications and variations would present themselves to those of skill in the art without departing from the scope and spirit of this invention. In particular, it is to be understood that this invention is not limited to the particular methodology, protocols, and reagents described as such may vary, as will be appreciated by one of skill in the art.

1-52. (canceled)

53. A light-activatable 2-component dental whitening system comprising:
a first gel comprising at least one peroxide; and
a second gel comprising an orally compatible Activator Gel at a pH of less than about 5.5, said Activated Gel comprising at least one transition metal salt for catalyzing the decomposition of the peroxide in the presence of light.

54. The light-activatable 2-component dental whitening system of claim 53 further comprising at least one component selected from the group consisting of a gelling agent, a source of calcium, a source of phosphate, a gel stabilizing agent, a foaming agent, a pH adjusting agent, glycerine, an aliphatic polyol, water and mixtures thereof.

55. The light-activatable 2-component dental whitening system of claim 53 wherein the second gel comprising the orally compatible Activator Gel at a pH from about 2 to 7.

56. The light-activatable 2-component dental whitening system of claim 53 wherein the second gel comprising the orally compatible Activator Gel at a pH from about 3 to 6.

57. The light-activatable 2-component dental whitening system of claim 53 wherein the second gel comprising the orally compatible Activator Gel at a pH from about 3 to 5.5.

58. The light-activatable 2-component dental whitening system of claim 53 further comprises at least one component comprising glyconic acid, gallic acid or mixtures thereof.
